(19) United States
(12) Patent Application Publication
(10) Pub. No.: US 2026/0251744 A1
(43) Pub. Date: Aug. 27, 2026

(54) SYSTEMS AND METHODS FOR AUTOMATICALLY PREDICTING INVERSION TIME IN MAGNETIC RESONANCE IMAGING

(71) Applicant: GE Precision Healthcare LLC, Waukesha, WI (US)

(72) Inventors: Subhashis Banerjee, Bengaluru (IN); Sudhanya Chatterjee, Bengaluru (IN); Dattesh Dayanand Shanbhag, Bengaluru (IN)

(21) Appl. No.: 19/061,837

(22) Filed: Feb. 24, 2025

(51) Int. Cl. G01R 33/56; G01R 33/54

(52) U.S. Cl. CPC G01R 33/5602; G01R 33/543; G01R 33/5601; G01R 33/5608

(57) ABSTRACT

Systems and methods are provided for contrast scans. In one example, a system includes a display device, one or more processors, and memory storing instructions executable by the one or more processors to obtain a series of TI scout images, each TI scout image in the series of TI scout images obtained with a different inversion time; process at least a portion of each TI-scout image in the series of TI scout images to generate a similarity metric plot that depicts a degree of statistical similarity between each pair of TI scout images of the series of TI scout images in a region of interest (ROI); predict an inversion time or a range of inversion times based on the similarity metric plot; and display the predicted inversion time or the range of inversion times on the display device.

### TECHNICAL FIELD

The present description relates generally to medical imaging. More specifically, the present disclosure relates to contrast-enhanced magnetic resonance imaging.

### BACKGROUND

Magnetic resonance imaging (MRI) is a medical imaging modality that can create images of the inside of a human body without using x-rays or other ionizing radiation. An MRI scan typically includes a series of radiofrequency (RF) excitation pulses and magnetic field gradient pulses that are played out with specific timings and in a specific sequence to prepare contrast and encode spatial information into the signal to generate an image. To enhance certain anatomical features, some MRI scans may include the administration of a contrast agent to a subject being imaged.

### BRIEF DESCRIPTION

In one example, a system includes a display device, one or more processors, and memory storing instructions executable by the one or more processors to obtain a series of TI-scout images, each TI-scout image in the series of TI-scout images obtained with a different inversion time; process at least a portion of each TI-scout image in the series of TI-scout images to generate a similarity metric plot that depicts a degree of statistical similarity between each pair of TI-scout images of the series of TI-scout images in a region of interest (ROI); predict an inversion time or a range of inversion times based on the similarity metric plot; and display the predicted inversion time or the range of inversion times on the display device.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 is a block diagram of an MRI apparatus according to an embodiment of the disclosure;

FIG. 2 schematically shows an example scan control device of the MRI apparatus of FIG. 1;

FIG. 3 is a high-level flow chart of a method for a contrast scan, according to embodiments of the disclosure;

FIG. 4 is a flow chart of a method for predicting inversion time from a series of scout images, according to embodiments of the disclosure;

FIG. 5 schematically shows a process for generating cropped image frames from a series of scout images using a region of interest (ROI) detector, according to the method of FIG. 4;

FIG. 6 schematically shows a process for predicting an inversion time using the cropped image frames of FIG. 5 and a mask predictor, according to the method of FIG. 4;

FIG. 7 shows an example similarity metric plot generated from a series of scout images according to the processes of FIGS. 5 and 6;

FIG. 8 shows a range of predicted inversion times selected from the similarity metric plot of FIG. 7;

FIG. 9 shows a first example series of scout images and a predicted inversion time;

FIG. 10 shows a second example series of scout images and a predicted inversion time;

FIG. 11 is a flow chart illustrating an example method for training a mask predictor, according to embodiments of the disclosure; and

FIG. 12 schematically shows a process for training a mask predictor, according to the method of FIG. 11.

### DETAILED DESCRIPTION

The following description relates to medical imaging, and in particular contrast-enhanced magnetic resonance (MR). Contrast-enhanced MR imaging (MRI) scans include the administration of a paramagnetic contrast agent, such as gadolinium-based contrast agents, to an imaging subject in order to enhance contrast of various tissues of the imaging subject. Contrast-enhanced MRI scans may be deployed in order to visualize myocardial scarring/ischemia following a myocardial infarction, for example. These scans, referred to as delayed-enhancement cardiac MRI or myocardial delayed enhancement (MDE) MRI, allow visualization of areas of fibrosis/scarring, as the areas of scarring demonstrate greater contrast accumulation than adjacent normal tissue, which is represented as a region of high intensity signal with a shorter longitudinal relaxation (T1) time.

Conventionally, MDE scans are performed using inversion recovery (IR) gradient-echo (GE) sequences 10-15 minutes after contrast (e.g., gadolinium) administration. In the IR sequence, an IR pulse inverts longitudinal magnetization of different tissue types. The magnetization recovery is different in each tissue type. For example, the tissue types of primary interest in cardiac MRI are typically normal myocardium, infarcted myocardium (infarct), and blood. The recovery rate of a particular tissue can be represented as the inverse of its longitudinal recovery time T1. A time delay, referred as inversion time (TI), is inserted between the IR pulse and the data acquisition sequence, such that the magnetization of one of the tissues (e.g., normal myocardium in the specific case of cardiac MRI) is approximately zero at the time of acquisition. This is often referred to as “nulling” the particular tissue, and can generate improved T1 image contrast between the nulled tissue and other tissue types (such as infarcted myocardium).

During an MDE scan, a scan operator may set the inversion time for the tissue to be nulled. However, optimal inversion times may vary from patient to patient. To determine the appropriate inversion time for an individual, a TI scout series is obtained where each image in the series has a progressively larger TI. In practice, selection of TI is generally performed through visual inspection and selection of the optimal TI from the TI scout series. This approach is dependent on the skill of a technologist or physician to select the optimal inversion time, and such skilled clinicians may not be readily available outside of specialized centers. Further, motion artifacts or metal artifacts may make selection of the optimal TI challenging. As such, consistent selection of the inversion time tends to be a significant problem, especially when different technicians are selecting the TI. In practice, this problem tends to be a frequent cause of patient call-backs, thus delaying patient diagnosis and decreasing scanning efficiency.

Thus, methods have been developed to automatically select the optimal inversion time for a patient based on a TI scout series. One such method utilizes a sliding window approach to define subsets of image frames within the TI scout series. The subsets are processed using a deep learning based image classifier to identify spatial features within the image set that allow separation of the subsets into classes corresponding to “before” and “after” the point that corresponds to the position of the optimal image within the TI scout series. However, current methods for automated selection of inversion time suffer from various drawbacks. As one example, different imaging facilities may utilize different protocols for acquiring the TI scout series, resulting in differing numbers of image frames from scan to scan. This demands multiple passes of the sliding window to determine the optimal inversion time, which is inefficient. Moreover, disease related anomalies and imaging related artifacts (motion, metal) can cause variations in the imaging data and consequently the TI selection. Hence, a robust algorithm should have capabilities to address for such variations in the data. If a machine-learning based approach is being used to select the TI, all such variations (including different cardiac views) have to be explicitly included in the training pool, which enlarges the needed training data pool to the point that assembling and labeling the training data may be impractical. Further still, deep learning models that identify spatial features in the images are generally large and demand a high amount of computing resources.

Thus, according to embodiments disclosed herein, the above issues may be addressed by combining localization of the region to be assessed for determining the optimal TI point in the TI scout images (and generating cropped frames of just the localized region) and then converting the cropped frames into probability density functions (PDFs) and computing differences between frames as a measure of a selected statistical distance. This approach projects the image frame data into a more interpretable measurement space. The measurements in this space focus on the variations between frames, highlighting key transitions or nullification points more clearly than image data frames, while being less sensitive to imaging related artifacts in the image frame such as noise, metal artifacts, etc. The ground truth TI location may be highlighted as a binary mask. The task for any AI solution is to segment this mask and generate the optimal TI using the location of this mask along temporal direction. A shallow Encoder-Decoder network is employed to segment a unit region around the TI point in the temporal statistical distance map. This now becomes a “single-pass” optimal TI determination methodology; where the measurement space is used to determine TI in blood pool and myocardium, irrespective of the subjectivity of the TI-Scout protocol, cardiac view, artifacts in myocardium and blood pool regions, and/or patient condition variations.

The approach disclosed herein includes the following steps. First, the cardiac region is roughly localized on each TI scout image (which may be maximum intensity projection (MIP) images) using deep learning to create cropped temporal frames. Statistical distance score maps (referred to herein as similarity metric plots) are generated, which effectively measure affinity between the cropped TI frames, and any variation in these are likely to happen in the vicinity of the optimal TI region. Automated analysis of these maps provides the approximate region of “phase changes” comprising blood-pool to myocardium nulling to initial recovery. This analysis provides interpretable signal of similarity across frames and aids in understanding the model's decision-making process. This interpretability is often lacking in models using imaging data, where the relationship between inputs and predictions can be less transparent. Finally, the TI estimate from above process is consolidated with the system pulse sequence design (PSD) to provide the best possible TI time to the user. The PSD is a set of instructions to be executed by the MRI apparatus to carry out a scan, and thus consolidating the TI estimate with the system PSD may include generating a PSD for an MDE scan that includes the TI between the IR pulse and the data acquisition sequence, and the PSD is used to command the MRI apparatus to carry out the MDE scan. Thus, once the TI is predicted, the TI is entered as a value in a PSD for the MDE scan.

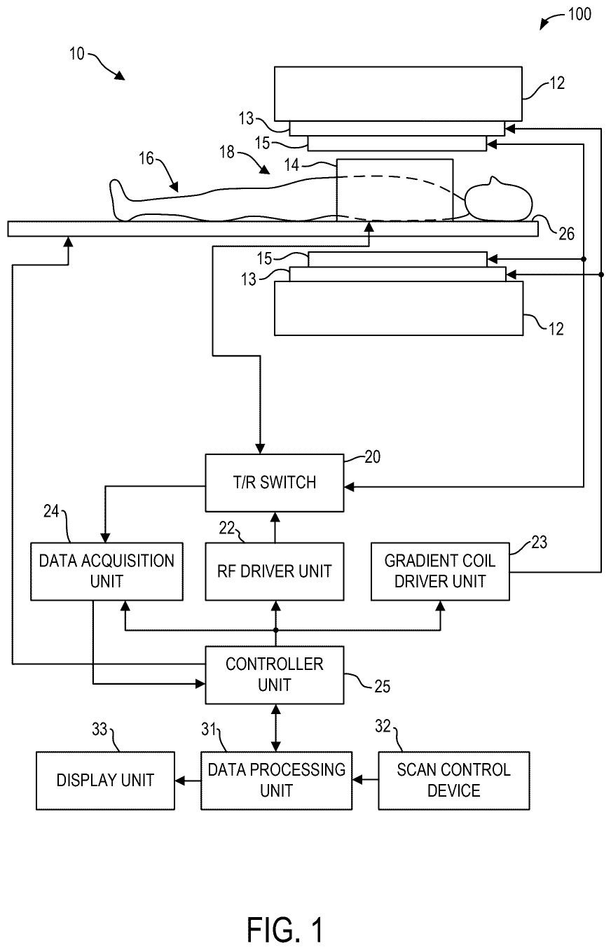

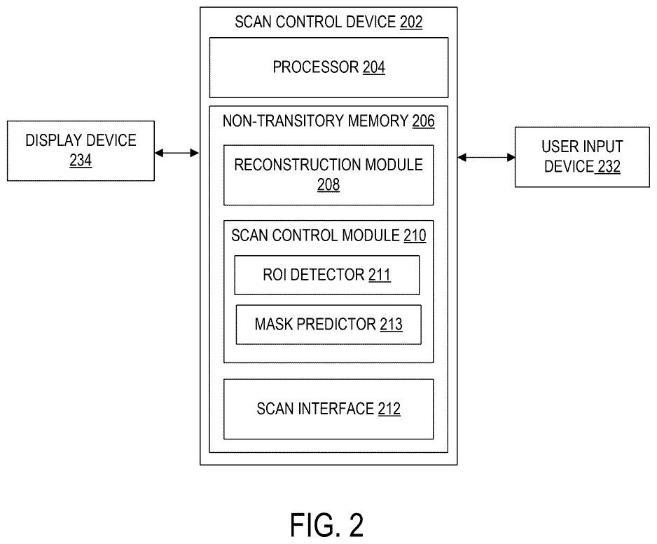

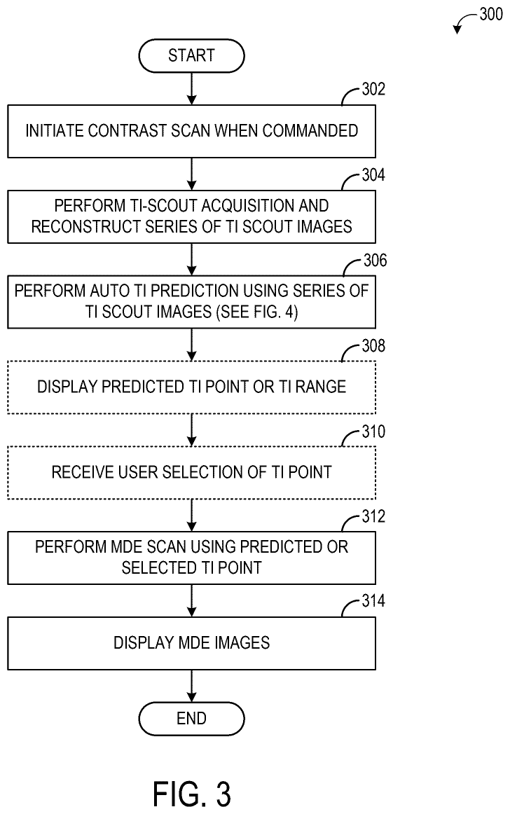

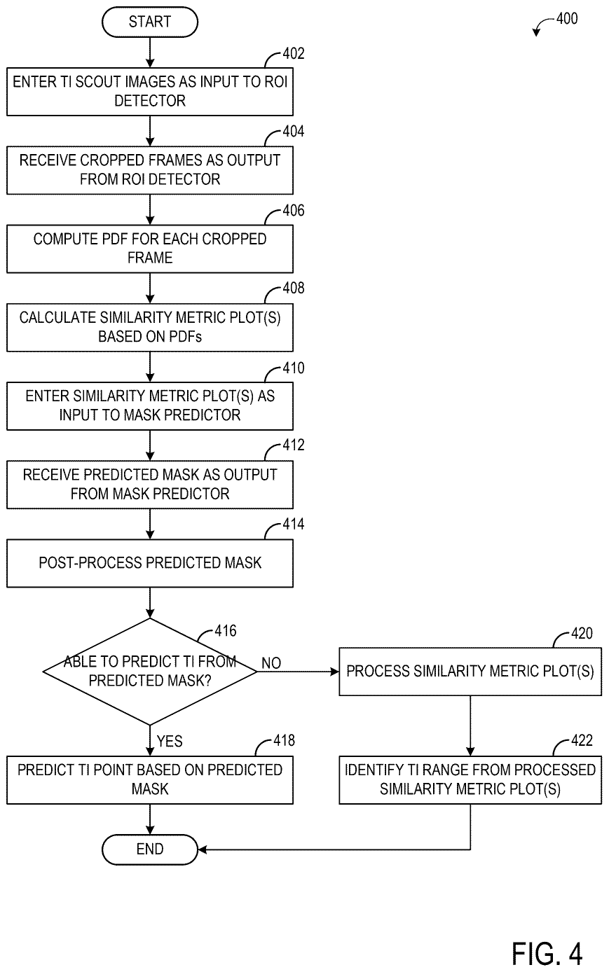

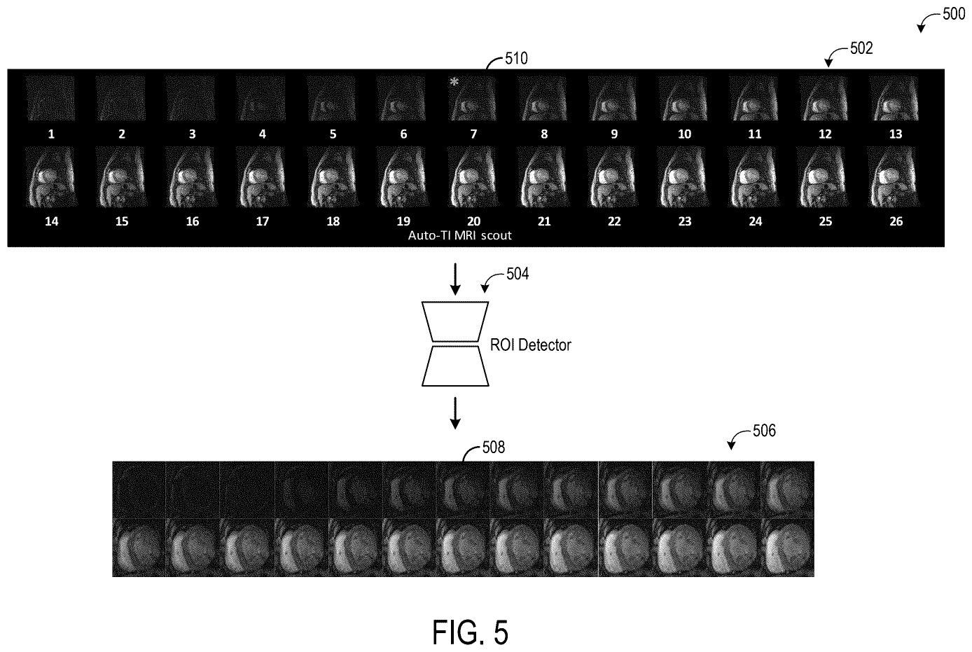

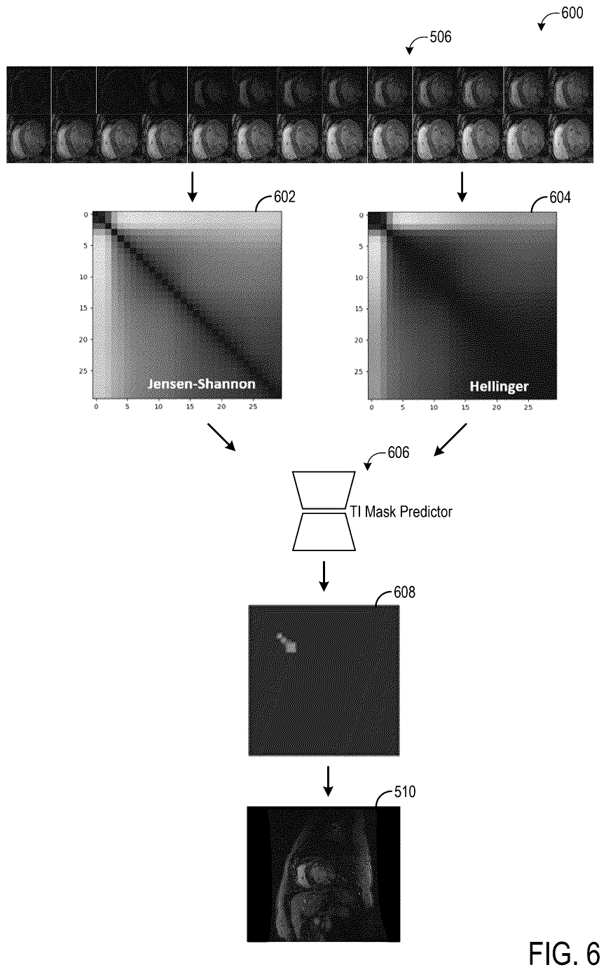

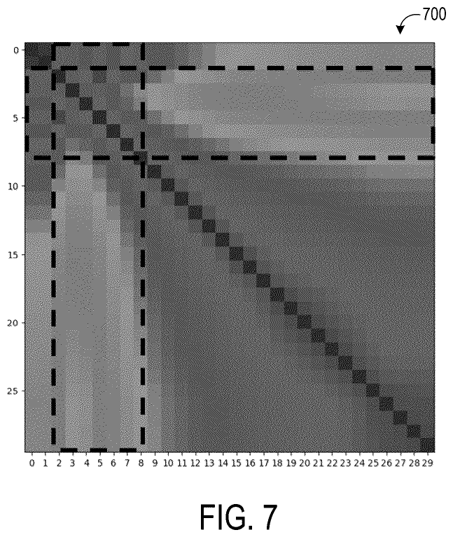

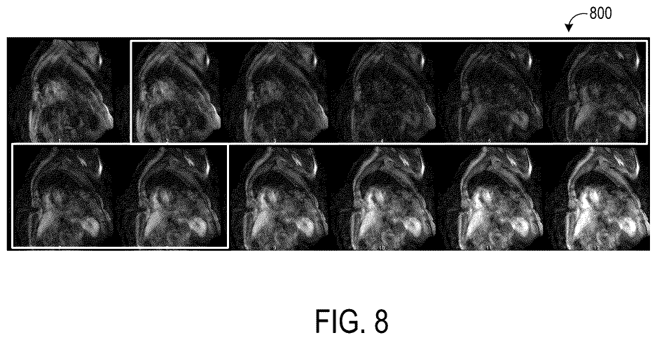

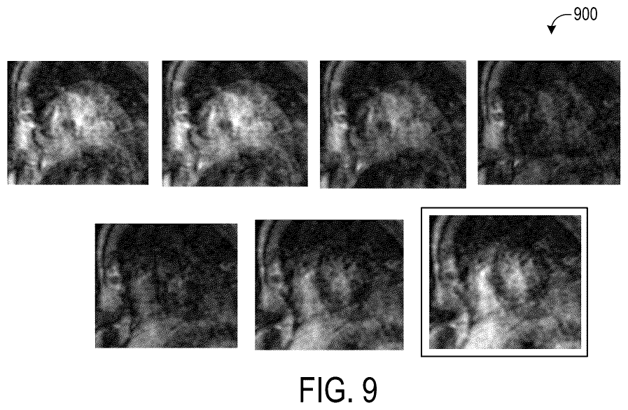

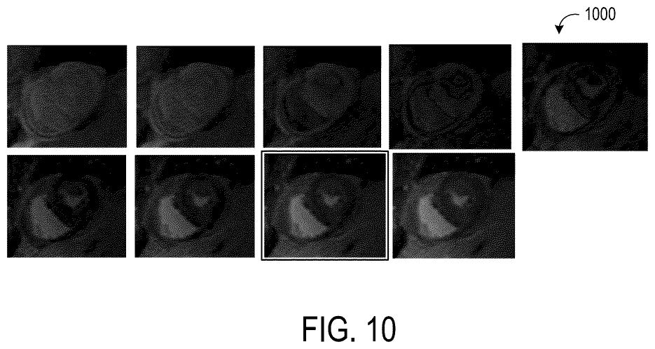

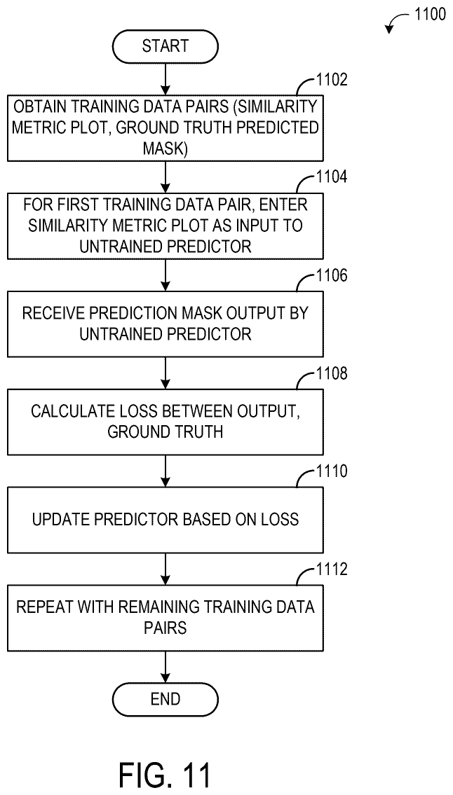

An example MRI apparatus that may be used to obtain images of an imaging subject during a contrast-enhanced MRI scan is shown in FIG. 1. The MRI apparatus may include a scan control device, such as the scan control device of FIG. 2, configured to process data from the MRI apparatus to form images, predict an optimal inversion time based on a TI scout series obtained with the MRI apparatus, command actions of the MRI apparatus (e.g., start the TI scout scan acquisitions, command the inversion time to be used in the MDE acquisitions, start the MDE acquisitions, and so forth), and display a scan interface to enable a user to monitor a contrast level of an imaging subject during the scan, set scan prescription parameters, confirm or select an optimal inversion time, and the like. The scan control device may carry out various methods to facilitate a contrast scan, such as methods shown in FIGS. 3 and 4, that includes prediction of an optimal inversion time, which are schematically illustrated in the processes of FIGS. 5 and 6. In some examples, rather than predict a single inversion time, the methods discussed herein may identify a range of inversion times, as shown in FIGS. 7 and 8. TI scout images that may be obtained with the MRI apparatus along with predicted TI frames identified according to the methods disclosed herein are shown in FIGS. 9 and 10. The predicted inversion time may be generated by a mask predictor that may be trained according to the method of FIG. 11.

FIG. 1 illustrates an MRI apparatus 10 (e.g., an MRI system) that includes a magnetostatic field magnet unit 12, a gradient coil unit 13, an RF coil unit 14, an RF body coil unit 15 (e.g., volume coil unit), a transmit/receive (T/R) switch 20, an RF driver unit 22, a gradient coil driver unit 23, a data acquisition unit 24, a controller unit 25, a patient bed or table 26, a data processing unit 31, a scan control device 32, and a display unit 33. In some embodiments, the RF coil unit 14 is a surface coil, which is a local coil typically placed proximate to the anatomy of interest of a subject 16. Herein, the RF body coil unit 15 is a transmit coil that transmits RF signals, and the local surface of the RF coil unit 14 receives the MR signals. As such, the transmit body coil (e.g., RF body coil unit 15) and the surface receive coil (e.g., RF coil unit 14) are separate but electromagnetically coupled components. The MRI apparatus 10 transmits electromagnetic pulse signals to the subject 16 placed in an imaging space 18 with a static magnetic field formed to perform a scan for obtaining magnetic resonance signals from the subject 16. One or more images of the subject 16 can be reconstructed based on the magnetic resonance signals thus obtained by the scan.

The magnetostatic field magnet unit 12 includes, for example, an annular superconducting magnet, which is mounted within a toroidal vacuum vessel. The magnet defines a cylindrical space surrounding the subject 16 and generates a constant primary magnetostatic field B₀.

The MRI apparatus 10 also includes a gradient coil unit 13 that forms a gradient magnetic field in the imaging space 18 so as to provide the magnetic resonance signals received by the RF coil arrays with three-dimensional positional information. The gradient coil unit 13 includes three gradient coil systems, each of which generates a gradient magnetic field along one of three spatial axes perpendicular to each other, and generates a gradient field in each of a frequency encoding direction, a phase encoding direction, and a slice selection direction in accordance with the imaging condition. More specifically, the gradient coil unit 13 applies a gradient field in the slice selection direction (or scan direction) of the subject 16, to select the slice; and the RF body coil unit 15 or the local RF coil arrays may transmit an RF pulse to a selected slice of the subject 16. The gradient coil unit 13 also applies a gradient field in the phase encoding direction of the subject 16 to phase encode the magnetic resonance signals from the slice excited by the RF pulse. The gradient coil unit 13 then applies a gradient field in the frequency encoding direction of the subject 16 to frequency encode the magnetic resonance signals from the slice excited by the RF pulse.

The RF coil unit 14 is disposed, for example, to enclose the region to be imaged of the subject 16. In some examples, the RF coil unit 14 may be referred to as the surface coil or the receive coil. In the static magnetic field space or imaging space 18 where a static magnetic field B₀ is formed by the magnetostatic field magnet unit 12, the RF body coil unit 15 transmits, based on a control signal from the controller unit 25, an RF pulse that is an electromagnet wave to the subject 16 and thereby generates a high-frequency magnetic field B₁. This excites a spin of protons in the slice to be imaged of the subject 16. The RF coil unit 14 receives, as a magnetic resonance signal, the electromagnetic wave generated when the proton spin thus excited in the slice to be imaged of the subject 16 returns into alignment with the initial magnetization vector. In some embodiments, the RF coil unit 14 may transmit the RF pulse and receive the MR signal. In other embodiments, the RF coil unit 14 may only be used for receiving the MR signals, but not transmitting the RF pulse.

The RF body coil unit 15 is disposed, for example, to enclose the imaging space 18, and produces RF magnetic field pulses orthogonal to the main magnetic field B₀ produced by the magnetostatic field magnet unit 12 within the imaging space 18 to excite the nuclei. In contrast to the RF coil unit 14, which may be disconnected from the MRI apparatus 10 and replaced with another RF coil unit, the RF body coil unit 15 is fixedly attached and connected to the MRI apparatus 10. Furthermore, whereas local coils such as the RF coil unit 14 can transmit to or receive signals from only a localized region of the subject 16, the RF body coil unit 15 generally has a larger coverage area. The RF body coil unit 15 may be used to transmit or receive signals to the whole body of the subject 16, for example. Using receive-only local coils and transmit body coils provides a uniform RF excitation and good image uniformity at the expense of high RF power deposited in the subject. For a transmit-receive local coil, the local coil provides the RF excitation to the region of interest and receives the MR signal, thereby decreasing the RF power deposited in the subject. It should be appreciated that the particular use of the RF coil unit 14 and/or the RF body coil unit 15 depends on the imaging application.

The T/R switch 20 can selectively electrically connect the RF body coil unit 15 to the data acquisition unit 24 when operating in receive mode, and to the RF driver unit 22 when operating in transmit mode. Similarly, the T/R switch 20 can selectively electrically connect the RF coil unit 14 to the data acquisition unit 24 when the RF coil unit 14 operates in receive mode, and to the RF driver unit 22 when operating in transmit mode. When the RF coil unit 14 and the RF body coil unit 15 are both used in a single scan, for example if the RF coil unit 14 is configured to receive MR signals and the RF body coil unit 15 is configured to transmit RF signals, then the T/R switch 20 may direct control signals from the RF driver unit 22 to the RF body coil unit 15 while directing received MR signals from the RF coil unit 14 to the data acquisition unit 24. The coils of the RF body coil unit 15 may be configured to operate in a transmit-only mode or a transmit-receive mode. The coils of the RF coil unit 14 may be configured to operate in a transmit-receive mode or a receive-only mode.

The RF driver unit 22 includes a gate modulator (not shown), an RF power amplifier (not shown), and an RF oscillator (not shown) that are used to drive the RF coils (e.g., RF body coil unit 15) and form a high-frequency magnetic field in the imaging space 18. The RF driver unit 22 modulates, based on a control signal from the controller unit 25 and using the gate modulator, the RF signal received from the RF oscillator into a signal of predetermined timing having a predetermined envelope. The RF signal modulated by the gate modulator is amplified by the RF power amplifier and then output to the RF body coil unit 15.

The gradient coil driver unit 23 drives the gradient coil unit 13 based on a control signal from the controller unit 25 and thereby generates a gradient magnetic field in the imaging space 18. The gradient coil driver unit 23 includes three systems of driver circuits (not shown) corresponding to the three gradient coil systems included in the gradient coil unit 13.

The data acquisition unit 24 includes a pre-amplifier (not shown), a phase detector (not shown), and an analog/digital converter (not shown) used to acquire the magnetic resonance signals received by the RF coil unit 14. In the data acquisition unit 24, the phase detector phase detects, using the output from the RF oscillator of the RF driver unit 22 as a reference signal, the magnetic resonance signals received from the RF coil unit 14 and amplified by the pre-amplifier, and outputs the phase-detected analog magnetic resonance signals to the analog/digital converter for conversion into digital signals. The digital signals thus obtained are output to the data processing unit 31.

The MRI apparatus 10 includes a table 26 for placing the subject 16 thereon. The subject 16 may be moved inside and outside the imaging space 18 by moving the table 26 based on control signals from the controller unit 25.

The controller unit 25 includes a computer and a recording medium on which a program to be executed by the computer is recorded. The program when executed by the computer causes various parts of the apparatus to carry out operations corresponding to pre-determined scanning. The recording medium may comprise, for example, a ROM, flexible disk, hard disk, optical disk, magneto-optical disk, CD-ROM, or non-volatile memory card. The controller unit 25 is connected to the scan control device 32 and processes the operation signals input to the scan control device 32 and furthermore controls the table 26, RF driver unit 22, gradient coil driver unit 23, and data acquisition unit 24 by outputting control signals to them. The controller unit 25 also controls, to obtain a desired image, the data processing unit 31 and the display unit 33 based on operation signals received from the scan control device 32.

The scan control device 32 includes user input devices such as a touchscreen, keyboard and a mouse. The scan control device 32 is used by an operator, for example, to input such data as an imaging protocol and to set a region where an imaging sequence is to be executed. The data about the imaging protocol and the imaging sequence execution region are output to the controller unit 25.

The data processing unit 31 includes a computer and a recording medium on which a program to be executed by the computer to perform predetermined data processing is recorded. The data processing unit 31 is connected to the controller unit 25 and performs data processing based on control signals received from the controller unit 25. The data processing unit 31 is also connected to the data acquisition unit 24 and generates spectrum data by applying various image processing operations to the magnetic resonance signals output from the data acquisition unit 24.

The display unit 33 includes a display device and displays an image on the display screen of the display device based on control signals received from the controller unit 25. The display unit 33 displays, for example, an image regarding an input item about which the operator inputs operation data from the scan control device 32. The display unit 33 also displays a two-dimensional (2D) slice image or three-dimensional (3D) image of the subject 16 generated by the data processing unit 31.

During an MRI scan using the MRI apparatus 10, a subject may be positioned within the imaging space 18 and an acquisition protocol may be carried out to obtain MR signals of the subject. The acquisition protocol may include a plurality of pulse sequences where in each pulse sequence, contrast is prepared via one or more RF pulses applied by the RF body coil unit 15 and the gradient coil unit 13 is controlled to spatially encode the resultant MR signals. The spatially-encoded MR signals are received by the RF coil unit 14 are digitized and stored in k-space. Thus, k-space data or a k-space dataset may refer to the raw MR signals prior to processing into an image. In some examples, one line of k-space may be filled with the raw MR signals per pulse sequence (also referred to as repetition time). In other examples, one line of k-space may be filled with the raw MR signals per echo, where more than one echo is generated per pulse sequence/repetition time. The k-space data may also be referred to as imaging data or MR data herein.

Referring to FIG. 2, scan control device 202 configured to control scan parameters of an MRI scan is shown. In some embodiments, scan control device 202 is incorporated into the MRI apparatus 10. For example, scan control device 202 may be provided in the MRI apparatus 10 as scan control device 32. In some embodiments, at least a portion of scan control device 202 is disposed at a device (e.g., edge device, server, etc.) communicably coupled to the MRI apparatus 10 via wired and/or wireless connections. In some embodiments, at least a portion of scan control device 202 is disposed at a separate device (e.g., a workstation) which can communicate with the controller unit of the MRI apparatus, for example. Scan control device 202 may be operably/communicatively coupled to a user input device 232 and a display device 234. In some examples, the user input device 232 may be the user input device of scan control device 32, explained above. Likewise, display device 234 may be the display unit 33 of MRI apparatus 10.

Scan control device 202 includes one or more processors, such as processor 204, configured to execute machine readable instructions stored in non-transitory memory 206. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206 may store a reconstruction module 208, a scan control module 210, and a scan interface 212. Reconstruction module 208 may be configured to reconstruct images from k-space data. In some examples, reconstruction module 208 may be the data processing unit 31 of FIG. 1, such that the data processing unit and scan control device are integrated into a single device. However, in other examples, reconstruction module 208 may be omitted and scan control device 202 may be in communication with the data processing unit 31 to obtain images for display.

Scan control module 210 may be configured to send commands to the MRI apparatus (e.g., to controller unit 25) in order to control aspects of a scan carried out by the MRI apparatus. Scan control module 210 may control aspects of the scan based on user input, which may be received via the scan interface 212, in some examples. For example, the scan control module 210 may process a series of TI scout images in order to predict an inversion time or a range of inversion times. As such, the scan control module 210 may store a region of interest (ROI) detector 211 and a mask predictor 213 that may be deployed in order to predict the inversion time, as will be explained in more detail below. The ROI detector 211 may be a deep learning-based model, such as a neural network (e.g., an encoder-decoder network). The mask predictor 213 may likewise be a deep learning-based model, such as a shallow encoder-decoder network. The scan interface 212 may display the predicted inversion time or the predicted range of inversion times. The scan interface 212 may include a scan prescription display panel via which a user may set parameters for the scan (e.g., a selected inversion time for an MDE scan).

In some embodiments, non-transitory memory 206 may include components disposed at two or more devices, which may be remotely located and/or configured for coordinated processing. In some embodiments, one or more aspects of non-transitory memory 206 may include remotely-accessible networked storage devices configured in a cloud computing configuration.

User input device 232 may comprise one or more of a touchscreen, a keyboard, a mouse, a trackpad, a motion sensing camera, or other device configured to enable a user to interact with and manipulate data within scan control device 202. In one example, user input device 232 may enable a user to make a selection of a scan protocol, adjust scan prescription settings, select or adjust a contrast-tracking region, and the like, as well as initiate, pause, and adjust scanning.

Display device 234 may include one or more display devices utilizing virtually any type of technology. In some embodiments, display device 234 may comprise a computer monitor, and may display MR images, including images reconstructed by reconstruction module 208. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view MRI images produced by an MRI system, and/or interact with various data stored in non-transitory memory 206.

It should be understood that scan control device 202 shown in FIG. 2 is for illustration, not for limitation. Another appropriate image processing system may include more, fewer, or different components.

FIG. 3 is a flowchart illustrating a high-level method 300 for a contrast scan, according to an embodiment of the disclosure. Method 300 may be implemented with the scan control device 202 of FIG. 2 in conjunction with the MRI apparatus 10 of FIG. 1. Method 300 may be carried out according to instructions stored in non-transitory memory and executed by a processor, such as non-transitory memory 206 and processor 204 of scan control device 202 of FIG. 2.

At 302, the contrast scan is initiated when commanded. For example, the contrast scan may be initiated in response to a user selection of a “Scan” button displayed on a display device. Once an operator of the MRI apparatus has been authenticated, a scan protocol interface may be displayed wherein the operator may indicate that a contrast scan is to be carried out on an imaging subject. In some examples, via the scan protocol interface, the operator may select a specific contrast-enhanced scan protocol, such as a myocardium delayed enhancement (MDE) scan, enter information about the imaging subject, etc. When the operator is ready to initiate the scan (e.g., the imaging subject is in the bore of the MRI apparatus), the operator may select the Scan button, which may both trigger initiation of the scan and cause a scan interface to be launched.

At 304, a TI-scout acquisition is performed and a series of TI scout images are reconstructed from MR data acquired with the MRI apparatus during the TI-scout acquisition. The series of TI scout images may be reconstructed via the reconstruction module 208. The series of TI scout images from the TI-scout acquisition may be viewed via the scan interface 212. The TI-scout acquisition may be carried out after a predetermined delay following administration of a contrast agent to the imaging subject, such as after 3 minutes, after 8 minutes, or after 10 minutes. The contrast agent may be a gadolinium-based contrast agent, at least in some examples. The TI-scout acquisition may be carried out according to a suitable protocol/prescription, such as Look-Locker, modified Look-Locker inversion recovery (MOLLI), or another sequence, where the TI is changed for each image acquisition (e.g., made progressively longer). The TI-scout acquisition may be gated (e.g., via an electrocardiogramsor) so that each acquisition is performed in the same cardiac cycle. The series of TI scout images may include a suitable number of images, such as in a range of 5-100 images, and may span a suitable range of inversion times, such as from 10 ms-1000 ms. The TI scout images may be in a suitable view, such as a mid-ventricular short-axis view, 4-chamber long-axis view, or another suitable view.

At 306, method 300 includes performing an auto-TI selection using the series of TI scout images. The auto-TI selection is described in more detail below with respect to FIG. 4. Briefly, the auto-TI selection includes localizing the cardiac region in each image of the series of TI scout images using an ROI detector; cropping each TI scout image of the series of TI scout images based on the localized cardiac region (such that image data outside of the localized/detected cardiac region is removed); computing a probability density function (PDF) for each cropped image; calculating one or more similarity metric plots based on the PDFs for the cropped images; and predicting an optimal TI based on the one or more similarity metric plots, which may be entered as input to a mask predictor trained to identify the optimal TI from the one or more similarity metric plots. For some TI scout images, the mask predictor may be unable to identify a single, optimal TI. Thus, in some examples, rather than predict one TI point, a range of possible TIs may be predicted by processing the similarity metric plot(s).

Accordingly, at 308, method 300 includes optionally displaying the predicted TI point (e.g., as predicted by the mask predictor) or the TI range (e.g., when the mask predictor cannot identify a single TI point). The predicted TI point or the TI range may be displayed via the scan interface 212. For example, the series of TI scout images may be displayed and the predicted TI point may be displayed by highlighting the TI scout image acquired with the predicted TI point, or the TI range may be displayed by highlighting the TI scout images acquired with TIs in the TI range. In other examples, the predicted TI point or TI range may be displayed as values (e.g., 350 ms or 200 ms-350 ms). At 310, method 300 includes optionally receiving a user selection of a TI point. In some examples, the user may confirm the auto-TI selection/mask predictor's predicted TI point or the user may select a TI point from the TI range. In some examples, the user may enter a TI value into a scan prescription interface displayed on the scan interface or select a TI value from a menu of the scan prescription interface, where the scan prescription interface includes various user interface elements (e.g., menus, buttons, text boxes) via which the user may set the prescription for a subsequent diagnostic scan. In still further examples, when a predicted TI point is identified, the predicted TI point may be automatically entered into the scan prescription interface and/or the predicted TI point may be included in the system PSD for the MDE scan.

At 312, method 300 includes performing the MDE scan using the predicted or selected TI point (e.g., the TI point selected by the user or the predicted TI point when automatically entered into the PSD for the scan). The MDE scan may include controlling the MRI apparatus to perform MDE acquisitions using an inversion recovery (IR) gradient-echo (GE) sequence, with the selected TI point being the delay time between the IR pulse and data acquisition (e.g., the center of the data acquisition window). The MDE scan may include acquisition of a plurality of short-axis views and/or long-axis views of the heart, with each slice/view obtained with the IR-GE sequence having the selected TI point. The MDE scan may be performed immediately after the TI-scout acquisition is performed (allowing for time to reconstruct the TI scout images and user selection of the TI point), or within a predetermined time from the TI-scout acquisition (e.g., 5 minutes) to ensure sufficient contrast levels during the MDE scan. At 314, MDE images reconstructed from the MDE acquisitions may be displayed via the scan interface. In some examples, the MDE images may be further processed and/or saved in long-term memory (e.g., in a picture archiving and communication system operably coupled to the scan control device). Method 300 then ends.

FIG. 4 is a method for predicting an inversion time from TI scout images, according to an embodiment of the disclosure. Method 400 may be implemented with the scan control device 202 of FIG. 2 in conjunction with the MRI apparatus 10 of FIG. 1. Method 400 may be carried out according to instructions stored in non-transitory memory and executed by a processor, such as non-transitory memory 206 and processor 204 of scan control device 202 of FIG. 2. In some examples, method 400 may be carried out as part of method 300, such as at 306 of method 300.

At 402, method 400 includes entering each TI scout image of the series of TI scout images as input to an ROI detector (e.g., ROI detector 211). The series of TI scout images may be acquired as explained above with respect to FIG. 3, and thus each TI scout image may be acquired with a different inversion time (TI) but may depict the same anatomy (e.g., the heart) in the same view (e.g., short-axis view) and at the same phase of the cardiac cycle. The ROI detector may be a neural network trained to identify a cardiac region of interest in each TI scout image. For example, the TI scout images may include the heart but may also include adjacent anatomy. The ROI detector may identify the cardiac ROI in each TI scout image and output a cropped frame, for each TI scout image, that includes only the cardiac ROI, with image data outside the cardiac ROI removed. The cardiac ROI may include all aspects of the heart visible in the TI scout images, or may only include certain features of the heart. At 404, method 400 includes receiving cropped frames as output from the ROI detector. In this way, the ROI detector may receive, as input, a TI scout image and may generate, as output, a cropped frame corresponding to the TI scout image that includes the cardiac ROI from the TI scout image and excludes image data of the TI scout image outside the cardiac ROI. Each TI scout image of the series of TI scout images may be input to the ROI detector such that a cropped frame is generated for each TI scout image of the series of TI scout images. Each cropped frame may have the same size and shape.

It is to be appreciated that the ROI detector is not trained to segment or otherwise identify individual cardiac features, such as the septum, endocardium, ventricular cavities, etc. Rather, the ROI detector may be trained with training data pairs, with each training data pair including a training TI scout image and a ground truth cropped frame that includes only the cardiac ROI from the training TI scout image. Each training TI scout image may of the same view, such that the ROI detector may only be trained to identify the cardiac ROI in one view. In such examples, when the trained ROI detector is employed during inference (e.g., as part of method 400), the ROI detector may be selected from among a plurality of ROI detectors based on the view of the TI scout images. In other examples, the ROI detector may be trained with training TI scout images in different views, such that one ROI detector is capable of identifying the cardiac ROI in any of a plurality of different views of the TI scout images.

At 406, a probability density function (PDF) is generated for each cropped frame to form a plurality of PDFs, each corresponding to a respective cropped frame (and hence respective TI scout image). The PDFs may be computed with a suitable approach, such as a histogram-based approach. For example, a PDF for a given cropped frame may be a normalized histogram that includes a plurality of bins, where each bin represents a pixel intensity value and the height of each bin is a probability of that pixel intensity value occurring within the cropped frame.

At 408, one or more similarity metric plots are calculated based on the plurality of PDFs. Each similarity metric plot may visualize a level of similarity between every two PDFs of the plurality of PDFs, where the level of similarity is calculated using a selected similarity metric. For example, a first similarity metric plot may be calculated using first similarity metric (such as a Hellinger distance) and a second similarity metric plot may be calculated using a second similarity metric (such as a Jensen-Shannon divergence). The Hellinger distance emphasizes distribution overlap between the two PDFs, while the Jensen-Shannon divergence captures information on divergence between the two PDFs but both similarity metrics provide a comprehensive view of the signal change between cropped frames. The Hellinger distance (d_{H}) for two PDFs P and Q (of two cropped frames) may be calculated according to the following equation 1:

$${d}_{H} = \sqrt{2 {\sum }_{i = 1}^{d} {( \sqrt{{P}_{i}} - \sqrt{{Q}_{i}} )}^{2}}$$

The Jensen-Shannon divergence (d_{JS}) for two PDFs P and Q (of two cropped frames) may be calculated according to the following equation 2:

$${d}_{JS} = \frac{1}{2} [ {\sum }_{i = 1}^{d} {P}_{i} ln ( \frac{2 {P}_{i}}{{P}_{i} + {Q}_{i}} ) + {\sum }_{i = 1}^{d} {Q}_{i} ln ( \frac{2 {Q}_{i}}{{P}_{i} + {Q}_{i}} ) ]$$

To generate a similarity metric plot using Hellinger distance, the Hellinger distance is calculated between every two PDFs of the plurality of PDFs. Each Hellinger distance may be visualized in the similarity metric plot as a color or grayscale value based on the value of that Hellinger distance. For example, lower Hellinger distances (which may indicate two PDFs, and hence two cropped frames, are similar) may be visualized with darker colors or darker grayscale values (e.g., black for the lowest Hellinger distances, then dark blue and dark purple or darker grays) and higher Hellinger distances may be visualized with lighter colors or lighter grayscale values (e.g., purple to light blue to light green to yellow, with yellow for the highest Hellinger distances, or medium to light gray with white or nearly white for the highest Hellinger distance). The similarity metric plot may be generated with a point (e.g., a block of pixels) for each combination of PDFs having the color or grayscale value assigned based on the Hellinger distance for that combination. A similarity metric plot using Jensen-Shannon divergence may be generated in a similar manner. An example of similarity metric plots using Hellinger distance and Jensen-Shannon divergence are shown in FIG. 6 and described in more detail below.

At 410, the similarity metric plot(s) are entered as input to a mask predictor (e.g., mask predictor 213). The mask predictor is trained to output a predicted mask that indicates a location of a predicted TI point based on the input similarity metric plot(s). Additional details about training of the mask predictor are provided below with respect to FIG. 11. In some examples, only one similarity metric plot may be entered as input to the mask predictor (e.g., generated with the first similarity metric or the second similarity metric). In other examples, two similarity metric plots may be entered as input to the mask predictor (e.g., one generated with the first similarity metric and the other generated with the second similarity metric), and the two similarity metric plots may be entered as input on different channels. Thus, at 412, method 400 includes receiving a predicted mask as output from the mask predictor. The predicted mask may have the same dimensions/scale as the similarity metric plot(s) and may include a point having a color or grayscale value that is different from the remaining points of the mask to indicate that point is the predicted TI point. However, in some examples, the predicted mask may include artifacts or background points, and thus to be able to identify the TI point from the predicted mask, the predicted mask may undergo post-processing, as indicated at 414. The post-processing may include adaptive thresholding to remove background noise, largest connected component identification, determination of a largest spot/point, and centroid placement based on the largest connected component. The centroid may be the TI point. For example, because the predicted mask in the same resolution as the similarity metric plots, the centroid may identify the region of the similarity metric plot(s) where a transition/signal change occurred, which can be mapped back to a cropped frame/TI scout image.

At 416, method 400 determines if a single TI point can be predicted from the predicted mask. If, after post-processing, multiple centroids are placed or a centroid cannot be placed, a single TI point may not be predicted from the predicted mask. Further, if the mask predictor cannot output a predicted mask, a single TI point may not be predicted from the predicted mask. If a single TI can be predicted from the predicted mask, method 400 proceeds to 418 to predict a TI point based on the predicted mask. The predicted TI point may then be displayed to a user, automatically entered into the scan prescription interface, and/or automatically included in the system PSD for the MDE scan, as explained above with respect to FIG. 3. Method 400 may end.

However, if a single TI point cannot be predicted from the predicted mask, method 400 proceeds to 420 to process the similarity metric plot(s) and, as indicated at 422, identify a TI range from the processed similarity metric plot(s). The processing may be performed by using Otsu thresholding on the similarity metric plot(s) and identifying the symmetric bands about the diagonal. The width of the bands provides the region of interest and hence the TI range. The TI range may then be displayed to a user so that the user can select a TI point from the TI scout images corresponding to the TI range, as explained above with respect to FIG. 3. Method 400 may end.

FIG. 5 schematically shows a process 500 for generating cropped image frames from a series of TI scout images using an ROI detector (e.g., ROI detector 211). Process 500 schematically depicts aspects of method 400, specifically the entering of the TI scout images to the ROI detector and the output of the cropped frames from the ROI predictor. As shown in FIG. 5, a series of TI scout images 502 of an imaging subject is obtained. Each TI scout image in the series of TI scout images 502 images the same anatomical region, in the same view/axis, during the same cardiac cycle phase. The only difference between the TI scout images of the series of TI scout images is the TI used in the pulse sequences during image acquisition. The TI (inversion time) may be progressively increased over the course of acquisition of the TI scout images. The TI scout images are numbered, with the first TI scout image (image 1) having the shortest TI and the last TI scout image (image 26 in the illustrated example) having the longest TI. The TI of each TI scout image is known and saved in memory along with the corresponding TI scout image, for example. Thus, the series of TI scout images 502 is ordered temporally based on inversion time, from shortest to longest TI. However, it is to be appreciated that other ways of ordering the TI scout images in the series of TI scout images is possible (such as reverse order, from longest TI to shortest TI), so long as the ordering is consistent.

Each TI scout image is entered as input to an ROI detector 504 (which is a non-limiting example of the ROI detector 211). As explained above with respect to FIGS. 2-4, the ROI detector 504 may be a neural network trained to identify a cardiac ROI in each input TI scout image and output a cropped frame of each input TI scout image. For example, the encoder of the ROI detector may extract features from the cropped frames into feature space and the decoder may process the extracted features to generate a segmentation mask of the cardiac ROI. The cardiac ROI may include the myocardium and blood pool, at least in some examples. Thus, after all images of the series of TI scout images 502 has been input to the ROI detector 504, a set of cropped frames 506 is obtained. Each cropped frame in the set of cropped frames 506 corresponds to a TI scout image from the series of TI scout images 502. For example, the seventh cropped frame 508 corresponds to the seventh TI scout image 510 in the series of TI scout images. e.g., the seventh cropped frame 508 is the cardiac ROI of the seventh TI scout image 510. The set of cropped frames 506 is ordered similarly to the series of TI scout images. By localizing the cardiac ROI and cropping the images so that only the cardiac region is present, the cropped frames (which are used to calculate a similarity metric plot from which the optimal TI point is predicted) are standardized such that performance is consistent irrespective of the field of view, patient size, or view changes.

FIG. 6 schematically shows a process 600 for predicting an inversion time from cropped image frames using similarity metrics and a mask predictor. Process 600 schematically depicts aspects of method 400, specifically the generation of the similarity metric plots from the set of cropped frames and TI point prediction from the predicted mask as output by the mask predictor. As shown in FIG. 6, the set of cropped frames 506 are processed to generate two similarity metric plots, according to the method described above with respect to FIG. 4. For example, each cropped frame is processed to generate a PDF of that cropped frame, to form a plurality of PDFs, each indicating a distribution of pixel intensity values in a corresponding cropped frame. A Jensen-Shannon divergence is calculated between every two PDFs of the plurality of PDFs. For example, a first Jensen-Shannon divergence is calculated for a first PDF of a first cropped frame (relative to the first PDF), a second Jensen-Shannon divergence is calculated for the first PDF and a second PDF for a second cropped frame, a third Jensen-Shannon divergence is calculated for the first PDF and a third PDF for a third cropped frame, and so forth. Each Jensen-Shannon divergence is plotted as a point (e.g., a pixel) on a first similarity metric plot 602, with the value of each divergence represented with a grayscale value. Each similarity metric plot may be an image. Because each PDF is compared with itself, the first similarity metric plot 602 includes a line of low divergence values that runs diagonally down the plot. Further, while the set of cropped frames 506 includes 26 frames, more or fewer cropped frames can be used to generate a similarity metric plot. To handle differing numbers of PDFs/cropped frames, the similarity metric plots may be transformed (e.g., via interpolation) into a standardized size (e.g., 30×30 points/pixels or 64×64 points/pixels). FIG. 6 also includes a second similarity metric plot 604 that is generated similarly to the first similarity metric plot 602, using Hellinger distances instead of Jensen-Shannon divergences. Further, while FIG. 6 depicts each similarity metric plot in grayscale, it is to be appreciated that each similarity metric plot may be visualized in color, as explained above with respect to FIG. 4.

One or both of the first similarity metric plot 602 and the second similarity metric plot 604 is entered as input to a TI mask predictor 606. The mask predictor 606 may be a non-limiting example of the mask predictor 213 and may comprise an encoder-decoder neural network configured to receive and process at least one similarity metric plot. The network can selectively process either a single similarity metric plot or multiple plots provided on different input channels, allowing flexible handling of both the first similarity metric plot 602 and the second similarity metric plot 604 as inputs. In some embodiments, the input similarity metric plots may be standardized to a predetermined size (e.g., 64×64, 32×32, or 128×128 points) through interpolation prior to processing.

The mask predictor 606's encoder-decoder neural network may be implemented with various architectural configurations to balance performance and computational efficiency. In some embodiments, the mask predictor 606 includes an input layer configured to accept standardized inputs of various dimensions (e.g., 32×32, 64×64, or 128×128 points) through interpolation, and may process these inputs using either single-channel or multi-channel configurations for handling different similarity metrics. The encoding path may comprise multiple convolutional layers with configurable kernel sizes and channel depths, various types of pooling layers for spatial dimension reduction, and different activation functions between layers. Optional normalization layers and configurable stride and padding options may also be employed. The decoding path may include upsampling layers or transpose convolution layers to restore spatial dimensions, with various approaches for feature map processing and refinement. In some implementations, skip connections may be incorporated between corresponding encoder and decoder layers to preserve fine spatial details. The mask predictor 606's network architecture may be configured with different numbers of encoding and decoding stages (e.g., two to four stages), varying numbers of kernels per layer (e.g., eight kernels, sixteen kernels, or other suitable numbers), and different activation functions such as ReLU or sigmoid, with the output layer typically employing sigmoid activation to generate pixel-wise regression values. Various optimization strategies may be implemented during training, such as different loss functions (e.g., dice loss, cross-entropy loss, or combinations thereof), attention mechanisms to focus on relevant image regions, or ensemble approaches combining predictions from multiple models. The specific architecture may be selected based on factors such as available computational resources, required inference speed, and desired prediction accuracy, while maintaining the network's ability to effectively process similarity metric plots and generate accurate predicted masks for TI point determination.

The mask predictor 606 generates a predicted mask 608 indicating regions likely to contain the predicted TI point. The predicted mask 608 has the same resolution as the input similarity metric plot(s) (e.g., 30×30 points, with each point being a pixel). Each pixel of the predicted mask 608 is assigned a regression value (or likelihood score) that indicates the likelihood that pixel belongs to the TI point. The regression values (which may be on a scale of 0-1, for example) are depicted in grayscale (or color) values. For example, pixels with a lower likelihood may be depicted in a first range of grayscale (or color) values (e.g., darker gray), while pixels with a higher likelihood may be depicted in a second range of grayscale (or color) values (e.g., lighter gray). In some implementations, the mask generation process may employ various techniques such as multi-scale processing to handle different spatial resolutions, attention mechanisms to focus on relevant image regions, or ensemble approaches combining predictions from multiple models trained with different loss functions (e.g., dice loss, cross-entropy loss, or combinations thereof).

Based on characteristics of the predicted mask 608, such as the presence of multiple high-likelihood regions or unclear boundaries, the mask predictor 606 may determine whether to output a single predicted TI point or indicate that multiple TI points should be considered. This flexibility allows the system to handle cases where a single optimal TI point may not be clearly identifiable due to imaging artifacts or pathological variations. The predicted mask undergoes post-processing steps including adaptive thresholding, connected component analysis, and centroid computation to determine the final predicted TI point location. Various post-processing approaches may be employed in different embodiments, such as: morphological operations to refine mask boundaries; weighted centroid calculation based on likelihood values; clustering algorithms to identify multiple potential TI points; or dynamic thresholding techniques that adapt to image characteristics. The post-processing may also include confidence scoring to assess prediction reliability, with different processing paths for high-confidence versus low-confidence predictions. In some implementations, temporal consistency checks may be performed across multiple cardiac cycles to validate TI point predictions.

As appreciated from FIG. 6, the predicted mask 608 may not cleanly identify one precise point of the similarity metric plot that corresponds to one PDF/cropped frame. Thus, additional processing of the predicted mask 608 may be performed, such as thresholding (e.g., to remove pixels with a regression values below a threshold, such as 0.5), identification of a largest connected component, centroid placement, etc. The final processed predicted mask may be used to determine the predicted TI point. In the example presented in FIG. 6, the predicted mask 608 identifies the seventh cropped frame 508/the seventh TI scout image 510 as the predicted TI point. Thus, as described previously, the number of frames can vary from TI scout to TI scout. To train the mask predictor 606, similarity metric plots are interpolated into a fixed size, for example, 64×64. In the interpolated space, the one-pixel mask (the ground truth mask) also gets rescaled based on the scaling factor derived from the original number of frames/resampled image dimension. Therefore, the mask predictor output can be a mask with more than one pixel, due to the interpolation/rescaling. After post-processing, the centroid or the center pixel is computed, which is further interpolated back into the original resolution.

Thus, the systems and methods described herein provide technical solutions to specific technical problems in the field of magnetic resonance imaging. Current MRI systems face the technical challenge of efficiently and accurately determining optimal inversion times across varying protocols, anatomical variations, and image artifacts. The disclosed solution described above with respect to FIGS. 1-6 improves the functioning of MRI systems by implementing a novel two-stage deep learning approach that reduces computational overhead while increasing robustness and accuracy.

Specifically, the ROI detector neural network is trained to efficiently process input images regardless of protocol variations, patient anatomical differences, or image artifacts-eliminating the need for protocol-specific training data or multiple processing passes. The shallow encoder-decoder architecture of the mask predictor network provides a technical improvement by reducing computational resource requirements compared to conventional deep learning approaches while maintaining accuracy. This architectural design allows the system to process TI scout images and generate predictions in near real-time during clinical workflows.

The technical solution includes specific improvements to neural network training and deployment that address the unique challenges of medical imaging applications. Rather than attempting to identify spatial features directly from raw scout images, which would require extensive training data covering all possible anatomical variations, the system first standardizes the input through ROI detection and PDF generation. This technical approach makes the system robust to variations in imaging protocols, patient anatomy, and image artifacts while reducing computational overhead. The result is a more efficient and reliable MRI system that can automatically determine optimal inversion times across a wide range of clinical scenarios without requiring protocol-specific training or multiple processing passes.

FIG. 7 shows an example similarity metric plot that may be used to predict a range of inversion times, as shown in FIG. 8. Specifically, FIG. 8 shows a series of TI scout images 800 that are processed as described above with respect to FIGS. 4-6 to generate a similarity metric plot 700 shown in FIG. 7. However, a single TI point is not identifiable from the similarity metric plot 700. Thus, the similarity metric plot 700 is processed to identify a TI range, shown by the dashed boxes on the similarity metric plot 700. For example, Otsu thresholding may be performed, which may identify one or more threshold pixel intensities that separate the pixels of the similarity metric plot 700 into two classes. The Otsu thresholding may result in identification of symmetric bands, as shown by the dashed boxes in FIG. 7. The width of the dashed boxes indicate that PDFs/cropped frames corresponding to the second through eighth TI scout images of the series of TI scout images 800 are identified in the TI range, which is indicated via the white boxes overlaid on the series of TI scout images.

In some examples, after obtaining a first series of TI scout images and predicting a first inversion time as described above, the system may obtain a second series of TI scout images (e.g., of a different patient), where each TI scout image in the second series is obtained with a different inversion time. The second series of TI scout images may be obtained from the same subject at a different time point or from a different subject. Similar to processing of the first series, at least a portion of each TI scout image in the second series is processed to generate a second similarity metric plot depicting statistical similarity between each pair of TI scout images of the second series in the ROI. The second similarity metric plot may be generated using the same approach described above—identifying and cropping to the cardiac ROI using the ROI detector, generating PDFs for the cropped frames, and calculating similarity metrics between PDFs to form the plot.

However, in some cases when the second similarity metric plot is input to the mask predictor, the mask predictor may be unable to predict a single inversion time point from the second similarity metric plot. This may occur, for example, if the second series of TI scout images exhibits significant motion artifacts or other image quality issues that make identification of a precise nulling point challenging. In such cases where a second predicted inversion time cannot be determined from the second similarity metric plot via the mask predictor, the system processes the second similarity metric plot to identify a range of predicted inversion times instead of a single point. The processing may include thresholding and analysis of transitions in the similarity metrics to identify a temporal window containing likely nulling points. The identified range of predicted inversion times is then displayed on the display device to guide manual refinement of TI selection. This provides a fallback mechanism when precise automated prediction is not possible, while still leveraging the information content in the similarity metric plot to assist the operator.

FIG. 9 shows a first example series of TI scout images 900 exhibiting motion artifacts. However, even with motion artifacts, the process described above with respect to FIGS. 4-6 is able to predict a TI point, herein the seventh TI scout image (shown by the box highlighting the seventh TI scout image). FIG. 10 shows a second example series of TI-scout images 1000 visualizing myocardium exhibiting a pathology. However, even with a pathology (which is a deviation from normal anatomy), the process described above with respect to FIGS. 4-6 is able to predict a TI point, herein the eighth TI scout image (shown by the box highlighting the eighth TI scout image). Thus, the approach disclosed herein is effective on challenging cases and does not demand representation of the various anomalies (e.g., motion artifacts, pathologies, etc.) in the training data, as in other approaches.

FIG. 11 illustrates a method 1100 for training a mask predictor to predict inversion times. Method 1100 may be carried out according to instructions stored in non-transitory memory and executed by a processor. In some examples, method 1100 may be carried out with instructions stored in non-transitory memory 206 as executed by processor 204 of scan control device 202 of FIG. 2. However, in other examples, method 1100 may be carried out on a remote/external computing device, such as a server, the cloud, etc. Method 1100 may be carried out to train the mask predictor described above with respect to FIGS. 2-6.

At 1102, method 1100 includes obtaining training data pairs. Each training data pair may include a similarity metric plot and a ground truth predicted mask. Each similarity metric plot may be generated as described above with respect to FIG. 4 and thus may represent relative similarity between every two PDFs of a respective plurality of PDFs, where each respective plurality of PDFs is generated from a respective set of cropped frames (which in turn are generated from a respective series of training TI scout images). The training TI scout images may be in one view or multiple views and may exhibit normal and/or abnormal anatomy, metal artifacts, and/or motion artifacts. Further, each series of training TI scout images may have the same or differing numbers of images. However, the mask predictor does not require any particular number of images in each series. Rather, as explained above, the similarity metric plots may be interpolated to transform the plots to a standard size (e.g., 30×30) for training. Additionally, the mask predictor does not require any particular coverage of anatomical views, pathologies, or artifacts. Thus, the training TI scout images used to train the mask predictor may be relatively easy to obtain and may be smaller in total number than other deep learning models used to automatically determine an optimal TI point.

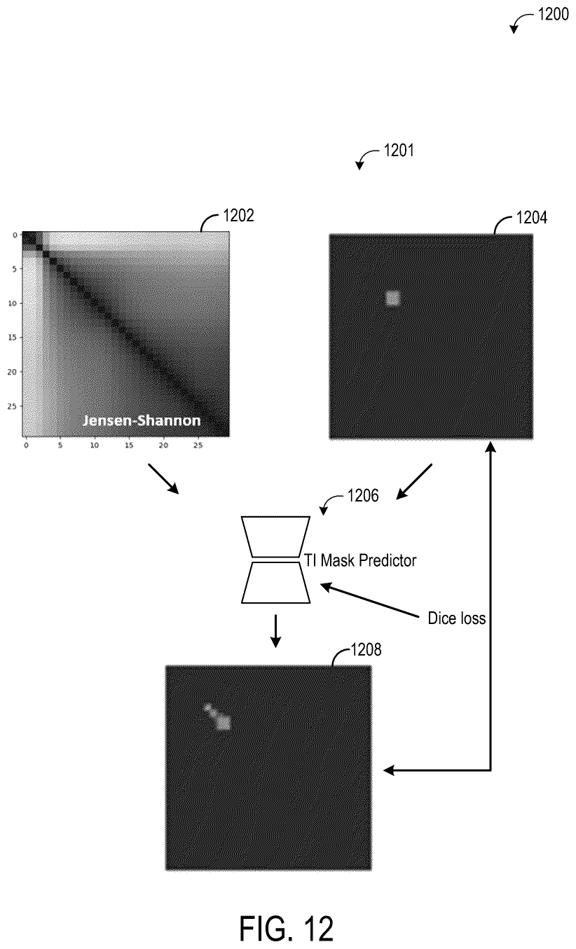

Each similarity metric plot may utilize the same similarity metric (e.g., Hellinger distance or Jensen-Shannon divergence). However, in some examples, the mask predictor may be trained to use both similarity metrics, and thus the training data pairs may include two similarity metric plots (one using a first similarity metric and one using a second similarity metric) and a ground truth predicted mask. Each ground truth predicted mask may be a binary mask having the same resolution as the similarity metric plot(s) and where the pixel of the optimal TI point is visualized with a first grayscale or color value and the remaining pixels are visualized with a second, different grayscale or color value. For example, FIG. 12 schematically shows a process 1200 for training a mask predictor using a first training data pair 1201. The first training data pair 1201 may include a similarity metric plot 1202 generated from a set of training TI scout images and a ground truth predicted mask 1204. The ground truth predicted mask 1204 may include a pixel colored differently than the rest of the pixels of the ground truth predicted mask 1204 to indicate the optimal TI point. The ground truth predicted mask 1204 is rescaled based on the scaling factor derived from the original number of frames of the set of training TI scout images so that the ground truth predicted mask 1204 is the same resolution as the similarity metric plot 1202. It is to be appreciated that the optimal TI point is indicated where the PDF of the frame corresponding to the optimal TI point (e.g., the seventh cropped frame) is compared to itself.

In some examples, each ground truth predicted mask may be generated based on user input. Specifically, one or more experts may review each series of training TI scout images to manually identify the optimal TI point, and a ground truth predicted mask may be generated that reflects the optimal TI point. In other examples, one or more ground truth predicted masks may be generated automatically. For example, one or more of the series of training TI scout images may be acquired as part of an MDE imaging exam(s) and the ground truth/optimal TI point may be set as the TI used for the subsequent MDE scan, for example.

At 1104, method 1100 includes, for a first training data pair, entering the similarity metric plot of the first training data pair as input to an untrained mask predictor. The untrained mask predictor may be a UNet (e.g., two encoding and decoding layers, eight kernels, one sigmoid output layer). If the mask predictor is to be trained based on two similarity metric plots, each of the two similarity metric plots of the first training data pair are entered as input (e.g., on different channels). At 1106, a predicted mask is received as output from the untrained mask predictor. At 1108, a loss is calculated between the predicted mask output by the mask predictor and the ground truth predicted mask of the first training data pair. The loss may be dice loss or another suitable loss. At 1110, the mask predictor is updated based on the loss. For example, the weights and/or biases of the mask predictor may be updated based on the loss. Referring again to FIG. 12, the similarity metric plot 1202 is entered as input to an untrained mask predictor 1206. The mask predictor 1206 outputs a predicted mask 1208. The predicted mask 1208 is compared to the ground truth predicted mask 1204 and a loss is calculated between the predicted mask 1208 and the ground truth predicted mask 1204. The loss is used to update the mask predictor 1206.

At 1112, the process is repeated with the remaining training data pairs. For example, the training data may include 500 training data pairs and the process of entering the similarity metric plot (of a given training data pair) as input to the mask predictor, receiving a predicted mask from the mask predictor, calculating a loss between the predicted mask and a ground truth predicted mask (of the given training data pair), and updating the mask predictor based on the loss may be performed for each training data pair, such that the loss is minimized. In some examples, each training similarity metric plot is generated from a respective training series of TI scout images using the same process described above for generating similarity metric plots during inference. Different training series of TI scout images may have different numbers of TI scout images—for example, a first training series may include 15 TI scout images while a second training series includes 26 TI scout images, reflecting variations in TI scout protocols across different imaging facilities. To accommodate these differences, training similarity metric plots generated from training series having different numbers of TI scout images are interpolated to a standard size. For example, a first training similarity metric plot generated from the first training series (having 15 TI scout images) and a second training similarity metric plot generated from the second training series (having 26 TI scout images) may each be interpolated to a standard 30×30 point size. This standardization ensures that all training similarity metric plots input to the mask predictor during training have consistent dimensions regardless of the number of TI scout images in the original training series from which they were generated. The standardization also allows the trained mask predictor to process similarity metric plots generated from any number of TI scout images during inference, providing flexibility to handle varying TI scout protocols.

The mask predictor may be considered trained once the process has been performed with each training data pair, or until the loss is sufficiently minimized. In some examples, the mask predictor may undergo 50-300 epochs of training using an Adam optimizer (with a learning rate=1e-4 in some examples) and dice loss. Method 1100 ends.

Thus, the embodiments disclosed herein allow for automated analysis of a series of TI scout images to predict an optimal TI point, where selected tissue, such as myocardium, is nulled, using a statistical-based approach that minimizes intensive processing of the images themselves. The approach includes generation of cropped frames from the series of TI scout images to focus analysis on only a region of interest (e.g., cardiac ROI where the myocardium is visualized), using a deep learning model. The cropped frames are converted to a plurality of PDFs that are compared via one or more similarity metrics (e.g., statistical distances) to form one or more similarity metric plots. The optimal TI point may be identified from the one or more similarity metric plots using a trained mask predictor, which may be a deep learning model trained to identify a particular transition point (e.g., myocardium nulling) in the similarity metric plots.

This approach may have several benefits. For example, the localization/cropping to form frames of just the cardiac region brings in standardization such that performance is consistent irrespective of field of view, patient size, or view changes. The optimal TI point is predicted in single shot manner without having to resort to multiple passes or tissue segmentation (as demonstrated by previous automated solutions) and thereby reducing compute time and robustness to disease and patient variations. The approach described herein provides reliable and consistent MDE imaging despite variations in TI-Scout protocol, cardiac views, or imaging artifacts. Even in case where the optimal TI point cannot be determined due to disease, artifact, etc., the similarity metric plots may convey information that can be used to guide the user to the approximate temporal location of the optimal TI point from the similarity metric plot(s), allowing for manual refinement without the need to scroll through all TI scout images.

The proposed solution provides single shot TI prediction capability by reducing the temporal image data to a 2D statistical measurement map (e.g., the similarity metric plot) and using deep learning (e.g., the mask predictor described above) to deduce the optimal TI point. The approach demands only a rough ROI around the myocardium, marked by a deep learning model (e.g., the ROI detector described above), without the need for fine segmentation of cardiac anatomies. There is no dependence on the number of frames or temporal scaling, and hence the approach can adapt to the subjectivity of the TI-scout protocols across sites. Further, the solution can detect if myocardium nullification has not occurred in the scout sequence (e.g., the series of TI scout images), and appropriate user feedback can be provided. Additionally, the solution supports multiple views, including axial, long axial views (4 chamber), and short-axis views and varying patient conditions. The approach disclosed herein reduces the temporal imaging data to a 2D statistical measurement map of frame-to-frame similarity and uses this map to compute TI location. Thus it overcomes need to train the deep learning model (e.g., mask predictor) for various orientations, patient conditions as needed in prior known solution and can handle temporal data of variable dimensions based on clinical protocols.

While the approach has been described herein as being specific for prediction of an optimal TI point in a series of TI scout images for MDE scanning, it is to be appreciated that the approach disclosed herein can be applied in other imaging contexts where frame-to-frame similarity and/or transition point determination are desired, such as dynamic perfusion imaging with injected contrast in MRI or computed tomography imaging.

The systems and methods disclosed herein provide several technical improvements to automated MRI inversion time prediction. By implementing the process of obtaining a series of TI scout images and processing them to generate a similarity metric plot depicting statistical similarity between image pairs in a region of interest (ROI), the system achieves more efficient and robust TI prediction while reducing computational overhead compared to conventional approaches. Specifically, the ROI detector's identification and cropping of each TI scout image to remove information outside the ROI provides technical advantages by normalizing the input data regardless of field of view, patient size, or cardiac view variations. This standardization eliminates the need for extensive training data covering all possible anatomical variations and imaging conditions. Further, the generation of probability density functions (PDFs) for the cropped frames and subsequent generation of similarity metric plots transforms the temporal imaging data into an interpretable 2D statistical measurement space. This transformation makes the analysis more robust against imaging artifacts and noise while highlighting key tissue nullification points more clearly than raw image data.

Additionally, the mask predictor, trained to output a predicted TI point based on the similarity metric plot, provides additional technical benefits by enabling single-pass TI prediction without requiring multiple sliding window operations or detailed tissue segmentation. This architectural choice significantly reduces computational complexity compared to deep learning models that must process full image spatial features. The system can handle variable numbers of input frames and different cardiac views without requiring protocol-specific training or model modifications.

The approach disclosed herein also provides improved artifact handling capabilities. By operating in the statistical measurement space rather than directly on image features through the generation and analysis of similarity metric plots, the system maintains reliable performance even in the presence of motion artifacts, metal artifacts, and pathological variations—conditions that typically degrade the performance of conventional image-based deep learning approaches. This robustness is achieved without requiring explicit training examples covering all possible artifact conditions, thereby dramatically reducing the training data requirements while improving generalization.

Furthermore, the system's ability to output either a predicted inversion time or a range of predicted inversion times based on the similarity metric plot represents a technical improvement in reliability and clinical workflow. The similarity metric visualization provides interpretable feedback about the prediction process, enabling informed manual refinement when needed—a capability typically lacking in black-box deep learning solutions.

These technical improvements result in a more efficient, robust and generalizable solution for automated TI prediction in MRI scanning while reducing computational resources, training data requirements, and sensitivity to imaging artifacts and protocol variations. The approach disclosed herein fundamentally transforms the technical nature of automated TI prediction from an image feature analysis problem to a more tractable statistical analysis task.

A technical effect of outputting a predicted inversion time or a range of predicted inversion times based on a similarity metric plot is the transformation of temporal MRI scout image data into a standardized statistical measurement space that enables robust and computationally efficient prediction of optimal inversion times while reducing sensitivity to imaging artifacts, protocol variations, and anatomical differences. By processing TI scout images to generate a similarity metric plot that depicts a degree of statistical similarity between each pair of TI scout images in a region of interest, the system provides a more tractable solution that reduces computational overhead compared to conventional image-based analysis methods while improving reliability by operating in a statistical domain rather than directly on image features, thereby providing a solution that is less dependent on extensive training data coverage of anatomical variations and imaging conditions.

The disclosure also provides support for a system, comprising: one or more processors, and memory storing instructions executable by the one or more processors to: obtain a series of TI scout images, each TI scout image in the series of TI scout images obtained with a different inversion time, process at least a portion of each TI scout image in the series of TI scout images to generate a similarity metric plot that depicts a degree of statistical similarity between each pair of TI scout images of the series of TI scout images in a region of interest (ROI), predict an inversion time based on the similarity metric plot, and output the predicted inversion time for inclusion in a pulse sequence design for a diagnostic scan. In a first example of the system, the system further comprises: a magnetic resonance imaging (MRI) apparatus, and wherein the instructions are further executable to command the MRI apparatus to carry out the diagnostic scan with the pulse sequence design that includes the predicted inversion time. In a second example of the system, optionally including the first example, processing at least a portion of each TI scout image in the series of TI scout images to generate the similarity metric plot comprises: identifying the ROI in each TI scout image using an ROI detector configured to crop each TI scout image to remove image information outside the ROI, and thereby form a set of cropped frames, calculating a probability density function (PDF) of each cropped frame, and generating the similarity metric plot from each PDF, wherein the similarity metric plot visualizes a similarity metric calculated between every two PDFs. In a third example of the system, optionally including one or both of the first and second examples, the similarity metric is a Hellinger distance or a Jensen-Shannon divergence. In a fourth example of the system, optionally including one or more or each of the first through third examples, predicting the inversion time based on the similarity metric plot comprises: entering the similarity metric plot as input to a mask predictor trained to output a predicted mask that identifies the predicted inversion time. In a fifth example of the system, optionally including one or more or each of the first through fourth examples, the series of TI scout images is a first series of TI scout images, the similarity metric plot is a first similarity metric plot, and the predicted inversion time is a first predicted inversion time, and wherein the instructions are further executable by the one or more processors to: obtain a second series of TI scout images, each TI scout image in the second series of TI scout images obtained with a different inversion time, process at least a portion of each TI scout image in the second series of TI scout images to generate a second similarity metric plot that depicts a degree of statistical similarity between each pair of TI scout images of the second series of TI scout images in the ROI, and determine that a second predicted inversion time cannot be predicted from the second similarity metric plot via the mask predictor, and in response, process the second similarity metric plot to identify a range of predicted inversion times and display the range of predicted inversion times on a display device. In a sixth example of the system, optionally including one or more or each of the first through fifth examples, processing the second similarity metric plot to identify the range of predicted inversion times comprises performing Otsu thresholding to identify one or more bands on the similarity metric plot, the one or more bands corresponding to the range of predicted inversion times.

The disclosure also provides support for a method, comprising: obtaining a series of TI scout images, each TI scout image in the series of TI scout images obtained with a different inversion time, processing at least a portion of each TI scout image in the series of TI scout images to generate a similarity metric plot that depicts a degree of statistical similarity between each pair of TI scout images of the series of TI scout images in a region of interest (ROI), predicting an inversion time or a range of predicted inversion times based on the similarity metric plot, and outputting the predicted inversion time or the range of predicted inversion times for inclusion in a pulse sequence design for a diagnostic scan and/or for display on a display device. In a first example of the method, the method further comprises: commanding a magnetic resonance imaging (MRI) apparatus to carry out the diagnostic scan with pulse sequence design that includes the predicted inversion time. In a second example of the method, optionally including the first example, the method further comprises: commanding a magnetic resonance imaging (MRI) apparatus to carry out the diagnostic scan with the pulse sequence design that includes a selected inversion time selected from the range of predicted inversion times. In a third example of the method, optionally including one or both of the first and second examples, processing at least a portion of each TI scout image in the series of TI scout images to generate the similarity metric plot comprises: identifying the ROI in each TI scout image using an ROI detector configured to crop each TI scout image to remove image information outside the ROI, and thereby form a set of cropped frames, calculating a probability density function (PDF) of each cropped frame, and generating the similarity metric plot from each PDF, wherein the similarity metric plot visualizes a similarity metric calculated between every two PDFs. In a fourth example of the method, optionally including one or more or each of the first through third examples, the similarity metric is a Hellinger distance or a Jensen-Shannon distance. In a fifth example of the method, optionally including one or more or each of the first through fourth examples, predicting the inversion time or the range of predicted inversion times based on the similarity metric plot comprises: entering the similarity metric plot as input to a mask predictor trained to output a predicted mask that identifies the predicted inversion time, if the predicted inversion time is identifiable from the predicted mask, outputting the predicted inversion time for inclusion in the pulse sequence design for the diagnostic scan, and if the predicted inversion time is not identifiable from the predicted mask, processing the similarity metric plot to identify the range of predicted inversion times and outputting the range of predicted inversion times for display on the display device.

The disclosure also provides support for a system, comprising: one or more processors, and memory storing instructions executable by the one or more processors to: obtain a series of TI scout images, each TI scout image in the series of TI scout images obtained with a different inversion time, process each TI scout image in the series of TI scout images with a region of interest (ROI) detector to generate a set of cropped frames, each cropped frame visualizing only an ROI in a corresponding TI scout image, generate a similarity metric plot that depicts a degree of statistical similarity between each pair of cropped frames of the set of cropped frames, process the similarity metric plot with a mask predictor trained to output a predicted TI point based on the similarity metric plot, and output a pulse sequence design for a diagnostic scan that includes the predicted TI point. In a first example of the system, the mask predictor is trained to output a predicted mask that identifies the predicted TI point, and wherein the mask predictor is trained with a plurality of training data pairs, each training data pair comprising a training similarity metric plot and a ground truth predicted mask. In a second example of the system, optionally including the first example, each training similarity metric plot is generated from a respective training series of TI scout images, wherein a first training series of TI scout images has a different number of TI scout images than a second training series of TI scout images, and wherein a first training similarity metric plot generated from the first training series of TI scout images and/or a second training similarity metric plot generated from the second training series of TI scout images is interpolated to a standard size, such that the first training similarity metric plot and the second training similarity metric plot are each in the standard size. In a third example of the system, optionally including one or both of the first and second examples, generating the similarity metric plot comprises calculating a probability density function (PDF) of each cropped frame, and generating the similarity metric plot from each PDF, wherein the similarity metric plot visualizes a similarity metric calculated between every two PDFs. In a fourth example of the system, optionally including one or more or each of the first through third examples, the similarity metric is a Hellinger distance or a Jensen-Shannon divergence. In a fifth example of the system, optionally including one or more or each of the first through fourth examples, the ROI is a cardiac ROI, wherein the diagnostic scan in a myocardial delayed enhancement scan, and wherein the pulse sequence design includes a time delay between an inversion recovery pulse and a data acquisition sequence that is equal to the predicted TI point. In a sixth example of the system, optionally including one or more or each of the first through fifth examples, the mask predictor comprises: an encoder-decoder neural network configured to: receive at least one similarity metric plot as input, process the at least one similarity metric plot to generate a predicted mask indicating regions likely to contain the predicted TI point, wherein the predicted mask is processed to determine a location of the predicted TI point, and wherein the system is configured to selectively output either a single predicted TI point or an indication that multiple TI points should be considered based on characteristics of the predicted mask.

As used herein, the term “approximately” is construed to mean plus or minus five percent of the range unless otherwise specified.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A system, comprising:
one or more processors; and
memory storing instructions executable by the one or more processors to:
obtain a series of TI scout images, each TI scout image in the series of TI scout images obtained with a different inversion time;
process at least a portion of each TI scout image in the series of TI scout images to generate a similarity metric plot that depicts a degree of statistical similarity between each pair of TI scout images of the series of TI scout images in a region of interest (ROI);
predict an inversion time based on the similarity metric plot; and
output the predicted inversion time for inclusion in a pulse sequence design for a diagnostic scan.

2. The system of claim 1, further comprising a magnetic resonance imaging (MRI) apparatus, and wherein the instructions are further executable to command the MRI apparatus to carry out the diagnostic scan with the pulse sequence design that includes the predicted inversion time.

3. The system of claim 1, wherein processing at least a portion of each TI scout image in the series of TI scout images to generate the similarity metric plot comprises:
identifying the ROI in each TI scout image using an ROI detector configured to crop each TI scout image to remove image information outside the ROI, and thereby form a set of cropped frames;
calculating a probability density function (PDF) of each cropped frame; and
generating the similarity metric plot from each PDF, wherein the similarity metric plot visualizes a similarity metric calculated between every two PDFs.

4. The system of claim 3, wherein the similarity metric is a Hellinger distance or a Jensen-Shannon divergence.

5. The system of claim 1, wherein predicting the inversion time based on the similarity metric plot comprises:
entering the similarity metric plot as input to a mask predictor trained to output a predicted mask that identifies the predicted inversion time.

6. The system of claim 5, wherein the series of TI scout images is a first series of TI scout images, the similarity metric plot is a first similarity metric plot, and the predicted inversion time is a first predicted inversion time, and wherein the instructions are further executable by the one or more processors to:
obtain a second series of TI scout images, each TI scout image in the second series of TI scout images obtained with a different inversion time;
process at least a portion of each TI scout image in the second series of TI scout images to generate a second similarity metric plot that depicts a degree of statistical similarity between each pair of TI scout images of the second series of TI scout images in the ROI; and
determine that a second predicted inversion time cannot be predicted from the second similarity metric plot via the mask predictor, and in response, process the second similarity metric plot to identify a range of predicted inversion times and display the range of predicted inversion times on a display device.

7. The system of claim 6, wherein processing the second similarity metric plot to identify the range of predicted inversion times comprises performing Otsu thresholding to identify one or more bands on the similarity metric plot, the one or more bands corresponding to the range of predicted inversion times.

8. A method, comprising:
obtaining a series of TI scout images, each TI scout image in the series of TI scout images obtained with a different inversion time;
processing at least a portion of each TI scout image in the series of TI scout images to generate a similarity metric plot that depicts a degree of statistical similarity between each pair of TI scout images of the series of TI scout images in a region of interest (ROI);
predicting an inversion time or a range of predicted inversion times based on the similarity metric plot; and
outputting the predicted inversion time or the range of predicted inversion times for inclusion in a pulse sequence design for a diagnostic scan and/or for display on a display device.

9. The method of claim 8, further comprising commanding a magnetic resonance imaging (MRI) apparatus to carry out the diagnostic scan with pulse sequence design that includes the predicted inversion time.

10. The method of claim 8, further comprising commanding a magnetic resonance imaging (MRI) apparatus to carry out the diagnostic scan with the pulse sequence design that includes a selected inversion time selected from the range of predicted inversion times.

11. The method of claim 8, wherein processing at least a portion of each TI scout image in the series of TI scout images to generate the similarity metric plot comprises:
identifying the ROI in each TI scout image using an ROI detector configured to crop each TI scout image to remove image information outside the ROI, and thereby form a set of cropped frames;
calculating a probability density function (PDF) of each cropped frame; and
generating the similarity metric plot from each PDF, wherein the similarity metric plot visualizes a similarity metric calculated between every two PDFs.

12. The method of claim 11, wherein the similarity metric is a Hellinger distance or a Jensen-Shannon distance.

13. The method of claim 8, wherein predicting the inversion time or the range of predicted inversion times based on the similarity metric plot comprises:
entering the similarity metric plot as input to a mask predictor trained to output a predicted mask that identifies the predicted inversion time;
if the predicted inversion time is identifiable from the predicted mask, outputting the predicted inversion time for inclusion in the pulse sequence design for the diagnostic scan; and
if the predicted inversion time is not identifiable from the predicted mask, processing the similarity metric plot to identify the range of predicted inversion times and outputting the range of predicted inversion times for display on the display device.

14. A system, comprising:
one or more processors; and
memory storing instructions executable by the one or more processors to:
obtain a series of TI scout images, each TI scout image in the series of TI scout images obtained with a different inversion time;
process each TI scout image in the series of TI scout images with a region of interest (ROI) detector to generate a set of cropped frames, each cropped frame visualizing only an ROI in a corresponding TI scout image;
generate a similarity metric plot that depicts a degree of statistical similarity between each pair of cropped frames of the set of cropped frames;
process the similarity metric plot with a mask predictor trained to output a predicted TI point based on the similarity metric plot; and
output a pulse sequence design for a diagnostic scan that includes the predicted TI point.

15. The system of claim 14, wherein the mask predictor is trained to output a predicted mask that identifies the predicted TI point, and wherein the mask predictor is trained with a plurality of training data pairs, each training data pair comprising a training similarity metric plot and a ground truth predicted mask.

16. The system of claim 15, wherein each training similarity metric plot is generated from a respective training series of TI scout images, wherein a first training series of TI scout images has a different number of TI scout images than a second training series of TI scout images, and wherein a first training similarity metric plot generated from the first training series of TI scout images and/or a second training similarity metric plot generated from the second training series of TI scout images is interpolated to a standard size, such that the first training similarity metric plot and the second training similarity metric plot are each in the standard size.

17. The system of claim 14, wherein generating the similarity metric plot comprises calculating a probability density function (PDF) of each cropped frame; and
generating the similarity metric plot from each PDF, wherein the similarity metric plot visualizes a similarity metric calculated between every two PDFs.

18. The system of claim 17, wherein the similarity metric is a Hellinger distance or a Jensen-Shannon divergence.

19. The system of claim 14, wherein the ROI is a cardiac ROI, wherein the diagnostic scan in a myocardial delayed enhancement scan, and wherein the pulse sequence design includes a time delay between an inversion recovery pulse and a data acquisition sequence that is equal to the predicted TI point.

20. The system of claim 14, wherein the mask predictor comprises:
an encoder-decoder neural network configured to:
receive at least one similarity metric plot as input;
process the at least one similarity metric plot to generate a predicted mask indicating regions likely to contain the predicted TI point;
wherein the predicted mask is processed to determine a location of the predicted TI point; and
wherein the system is configured to selectively output either a single predicted TI point or an indication that multiple TI points should be considered based on characteristics of the predicted mask.
